(19) United States
(12) Patent Application Publication
(10) Pub. No.: US 2009/0092975 A1
(43) Pub. Date: Apr. 9, 2009

(54) SELECTABLE MARKER

(75) Inventor: Malcolm Stratford, Bedfordshire (GB)

Correspondence Address: CLARK & ELBING LLP, 101 FEDERAL STREET, BOSTON, MA, 02110, US

(73) Assignee: Mologic Ltd, Sharnbrook (GB)

(21) Appl. No.: 12/097,655

(22) PCT Filed: Dec. 18, 2006
(86) PCT No.: PCT/GB2006/004757

§ 371 Date: Jul. 25, 2008

(30) Foreign Application Priority Data
Dec. 16, 2005 (GB) 0525674.8

(51) Int. Cl. C12Q 1/68; C07H 21/00; C12N 15/63; C12N 9/00

(52) U.S. Cl. 435/6; 536/23.2; 435/320.1; 435/183

(57) ABSTRACT

A nucleic acid sequence encoding a decarboxylation enzyme E.G. PAD1 is used as a selectable marker in a recombinant organism. A weak acid is used as the selecting agent.

The present invention relates to the use of a nucleic acid sequence encoding a selectable marker. The invention also relates to a method of selecting a recombinantly transformed micro organism, such as a prokaryote or yeast. In addition, the present invention relates to a vector comprising the nucleic acid sequence and a cell comprising such a vector.

A common practice in the field of biotechnology is to “transform” a host cell with exogenous nucleic acid sequences. In some instances, this is so as to confer a particular trait on the host cell, that is to say a function which is carried out by a protein encoded by the exogenous sequence. An example of this would be the transformation of a host cell with a nucleic acid sequence encoding the luminescent protein luciferase which would confer the trait of luminescence on the host cells. In other instances, the exogenous nucleic acid sequence confers no trait on the host cell, as such, but is inserted into the cell as part of a larger genetic manipulation. An example of this would be when a host cell is transformed with a part of a putative gene library.

There are several known techniques for transforming host cells with exogenous nucleic acid sequences such as, for example, electroporation and microparticle bombardment. The problem with these known techniques is that they have a relatively low and variable probability of success and therefore the result of the transformation process is a mixture of cells, only some of which have been successfully transformed with the exogenous nucleic acid, and most of which have not.

In order to overcome this problem, it is a standard technique to transform host cells with a nucleic acid sequence encoding a selectable marker, in addition to the nucleic acid sequence encoding the exogenous sequence. Both sequences are provided on the same vector so that a cell will either be transformed with both sequences or will not be transformed at all. It is therefore possible to extrapolate that cells that have the activity conferred by the selectable marker are also positive for the exogenous sequence. For example, it is known to use as selectable markers nucleic acid sequences encoding a protein conferring antibiotic resistance, as used in Barthelmebs et al. (2000) Appl. Environ. Microbiol. 66 3368-3375. The cells which result from the transformation process are then exposed to the antibiotic, which is the “selecting agent”, at a concentration which inhibits any cells which are not transformed with the resistance sequence. However the antibiotic is rendered ineffective (or at least substantially ineffective) to the cells transformed with the antibiotic resistance sequence and the exogenous nucleic acid sequences. In this way, it is possible to enrich the mixture of cells resulting from the transformation process so that all, or almost all, of the surviving cells have been transformed with the exogenous nucleic acid sequence.

Unfortunately, there are several problems with using antibiotic resistance genes as selectable markers. First of all, there is a concern that genes encoding antibiotic resistance could accidentally become passed into micro organisms in the environment leading to strains of infectious pathogens which are immune to antibiotics. This concern is reflected in the need for risk assessments for the release of genetically modified organisms. Secondly, antibiotics, themselves, can be expensive and it is costly to administer them to samples of transformed micro organisms continually. It is particularly a problem because, even after the transformation process is complete, it is necessary to continue to expose the micro organisms to the antibiotic to ensure that any micro organisms which lose the vector containing the exogenous nucleic acid are inactivated. Furthermore, if antibiotics are used on a large scale then there are issues over their subsequent disposal. Thirdly, antibiotics can be degraded by heat and so it can be inconvenient to use antibiotics in microbiological protocols where the heating of samples is required. For example, any nutrient broths which will be used to grow the transformed cells will usually be heat sterilised prior to incubation with the host cells. Therefore, it is essential that the antibiotic is not added to the nutrient broth prior to heat sterilisation. Fourthly, antibiotic selection of yeast cells must normally be carried out at pH 6 to 7 in order for the antibiotic to be effective, at which pH bacteria are also capable of growing. Therefore, using antibiotic selection also requires sterilisation of equipment to ensure that bacteria do not infect media containing transformed yeast, which is sub-optimal for yeast growth.

Another problem occurs when the host cells are fungal cells such as yeast. Comparatively few antibiotics for fungi are known. Therefore, the usual technique for selectively marking fungal cells is to begin with cells which are auxotrophic mutants, in other words cells which have additional nutritional requirements to wild-type cells, due to the inability of the cell to synthesise a particular organic compound required for growth. The selectable marker that is used is a gene which confers the ability to grow even in the absence of the particular organic compound. Thus, when cells are grown in the absence of the organic compound, only those cells transformed with the selectable marker are capable of growing. The problem with this approach is that it is necessary to start the process with auxotrophic host cells. This requires either that the host cells are carefully selected in order to be of a strain which is naturally auxotrophic or mutant cells be prepared which are auxotrophic. WO2004/042036, which is incorporated herein by reference, discloses inter alia vectors for transforming yeast cells which are auxotrophic for uracil synthesis.

Hartl et al. (2005) Curr. Genet. 48 204-211 and Grimm et al. (1998) Mol. Gen. Genet. 215 81-86 describe a selection process where the host cell strains that contain the URA3 gene, encoding orotidine-5′-decarboxylase, do not survive in the selective medium 5-fluoroorotic acid. The mutant cells lacking orotidine-5′-decarboxylase are selected because they are auxotrophic for uridine. In theory, the strains that contain URA3 are discoverable by negative selection.

CA 2,350,328 A1 and U.S. Pat. No. 6,303,846 B1 describe an oxalate decarboxylase gene from fungi (Aspergillus) and its putative role in degrading oxalate, and assays of oxalate for its use as a selectable marker in plants only. The dicarboxylic acid, oxalic acid, is not toxic to other microorganisms. Therefore it could not be used as a selectable marker in bacteria and yeast.

The present invention seeks to alleviate one or more of the above problems.

The present invention is based on the realisation that nucleic acids encoding decarboxylation enzymes can be used as selectable markers and weak acids used as the selecting agent.

Most micro organisms comprise, as part of their genome, one or more endogenous decarboxylase genes. However, such endogenous decarboxylase enzymes are typically not expressed at sufficiently high levels to confer full resistance to weak acids. In this regard, although it is reported in Clausen et al (Gene, 142 (1994) 107-112) that the PAD1 gene confers resistance to cinnamic acid in Saccharomyces cerevisiae, it is thought by the present inventors that what was actually observed in the study was the sensitivity of mutant S. cerevisiae with reduced PAD activity to withstand low concentration levels of weak acid rather than that the PAD1 gene could actually confer full resistance to cinnamic acid.

According to one aspect of the present invention, there is provided the use of a nucleic acid sequence encoding a decarboxylation enzyme as a selectable marker in a recombinant micro organism.

According to another aspect of the present invention, there is provided a method of selecting a recombinantly transformed micro organism comprising the steps of:

a) transforming a micro organism with a selectable marker comprising a recombinant nucleic acid sequence encoding a decarboxylation enzyme such that the decarboxylation enzyme is expressed by the micro organism; and

b) exposing the micro organism to a weak acid or a salt thereof at a concentration capable of inactivating the micro organism without the selectable marker.

Conveniently, the weak acid has a pKa of between pH 2.5 and pH 5.5, preferably between pH 4 and pH 5.

Preferably, the weak acid is an acid selected from Table 1 or 2.

It is preferred that the weak acid is provided at a concentration of at least 0.075 mM. For example, the weak acid may be provided in the concentration range 0.15 to 0.225 mM or 0.075 to 0.11 mM.

Advantageously, step b) is carried out at less than pH 6, preferably less than pH 5, more preferably less than pH 4.

Alternatively, the weak acid is not more than 50% disassociated between pH2.5 and 5.5.

Preferably, the weak acid is monocarboxylic.

Conveniently, the weak acid has a structure in accordance with formula (I)

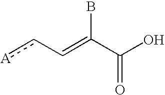

wherein [symbol] is:
(a) [symbol] alkyl optionally substituted; or
(b) an unsaturated aromatic group, optionally substituted with one or more hydrophobic groups; and
wherein B is hydrogen or a hydrophobic group.

Preferably, the unsaturated aromatic group is a phenyl group, a furanyl group, a thienyl group, pyrrole, cyclohexene, cyclopentene, cycloheptene or pyridine.

In some embodiments, the term “hydrophobic group” means a group that carries no charge at or near pH 7.0. In other embodiments, the term “hydrophobic group” means a “lipophilic group”, that is to say a group that is preferentially dissolved in lipid rather than water, i.e. Log Poct is greater than zero (Log Poct being the partition coefficient in octanol). Aside from hydroxy groups, the term includes, for example: —F; —Cl; —Br; —I; C₁₋₃ alkyl; C₁₋₃ alkoxy; mono- di- or tri-fluoro C₁₋₃ alkyl; mono- di- or tri-chloro C₁₋₃ alkyl; mono- di- or tri-bromo C₁₋₃ alkyl; and mono- di- or tri-iodo C₁₋₃ alkyl. Where the weak acid comprises a plurality of hydrophobic groups, they may each be the same or different.

Preferably, the salt is a sodium or potassium salt of the weak acid.

Advantageously, the micro organism is a prokaryote or lower eukaryote.

Conveniently, the micro organism is a yeast, preferably Saccharomyces cerevisiae.

Preferably, the nucleic acid sequence is part of a vector which further comprises a promoter for controlling expression of the decarboxylation enzyme.

According to another aspect of the present invention, there is provided a vector comprising a nucleic acid sequence encoding a decarboxylation enzyme and a constitutive promoter for controlling expression of the decarboxylation enzyme.

Advantageously, the promoter for controlling the expression of the decarboxylation enzyme is a recombinant promoter, preferably a constitutive promoter. By “constitutive promoter” is meant a promoter which is capable of effecting expression of a gene to which it is linked without being induced by another factor.

Conveniently, the promoter for controlling expression of the decarboxylation enzyme is a prokaryotic promoter and the vector further comprises a second promoter for controlling expression of the decarboxylation enzyme, the second promoter being a eukaryotic promoter.

Preferably the vector further comprises a multiple cloning site or an exogenous nucleic acid sequence.

Advantageously, the vector further comprises a secretion sequence operably linked to the multiple cloning site or exogenous nucleic acid.

Conveniently, the vector further comprises a prokaryotic origin of replication.

Preferably, the vector further comprises a eukaryotic autonomous replication sequence (ARS).

Advantageously, the vector further comprises a centromeric sequence.

Conveniently, the vector further comprises a homologous recombination sequence.

Preferably, the decarboxylation enzyme has at least 30% identity to SEQ. ID NO. 2.

Advantageously, the decarboxylation enzyme has at least 32%, 40%, 50%, 60%, 70%, 80%, 90% or 100% identity to SEQ. ID NO. 2.

According to a further aspect of the present invention, there is provided a recombinantly transformed cell comprising a vector as described above.

Conveniently, the vector is independent of the cell genome.

Alternatively, the vector is integrated into the cell genome.

Advantageously, the cell is a prokaryotic cell or a lower eukaryotic cell, preferably a yeast cell.

According to another aspect of the present invention there is provided the use of a decarboxylation enzyme as a selectable marker in a recombinant micro organism.

The term a “recombinant promoter” means a promoter other than the promoter that controls expression of the nucleic acid sequence encoding the decarboxylation enzyme as it exists in nature.

The term a “promoter for controlling the expression of a decarboxylation enzyme” means a promoter that effects expression of the enzyme in a host cell. A promoter is considered to have such an effect when its presence results in a higher level of expression of the enzyme in a host cell.

The term a “constitutive promoter” means a promoter that is unregulated and results in the expression of a constant level of enzyme or protein in a host cell, as opposed to an inducible promoter.

The term ‘constitutive promoter’ means a promoter that results in the unconditional expression of a protein or enzyme at a constant (maximal) level independently of growth stage, or environmental stimuli, as opposed to an inducible promoter.

The term “positive selectable marker” means a gene introduced into a cell that when expressed confers a trait or characteristic which permits the cell to survive better in a medium in which it would otherwise not survive or divide as frequently (for example, toxic selective medium), so allowing the positive identification of the surviving transformed or transfected cells which contain the vector with the marker gene.

In order that the present invention may be further understood and embodiments of the invention exemplified, embodiments will now be described with reference to the following figures in which:

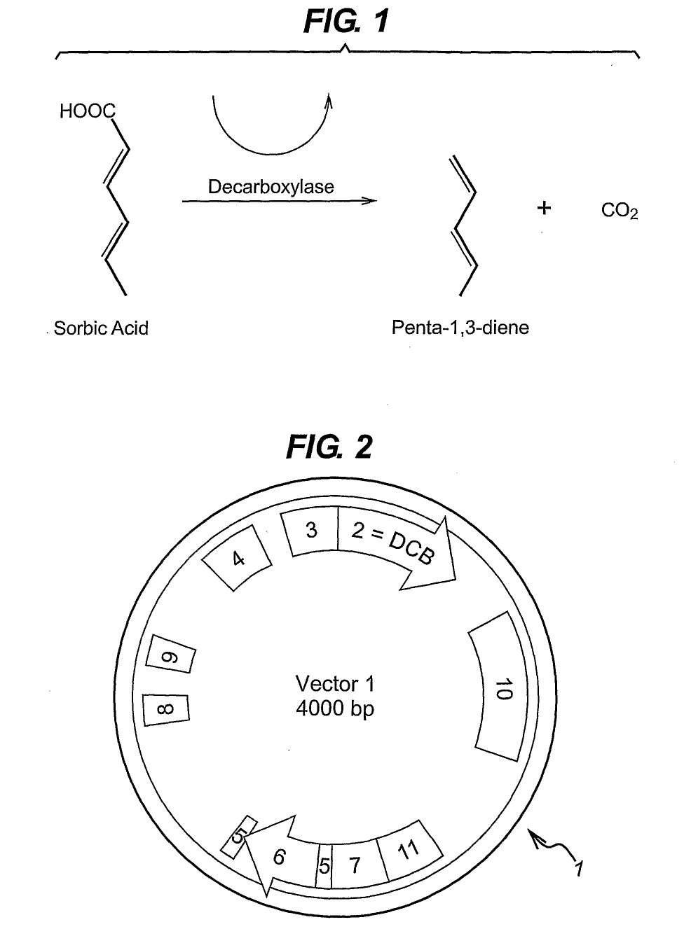

FIG. 1 is a reaction scheme showing the decarboxylation of sorbic acid;

FIG. 2 is a schematic diagram of a vector in accordance with one embodiment of the present invention;

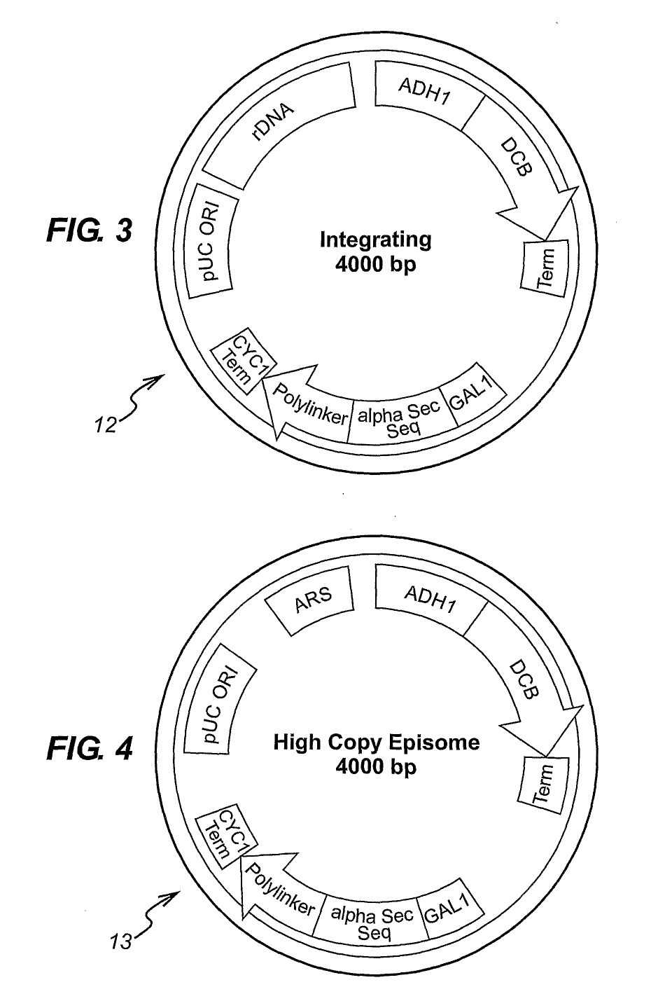

FIG. 3 is a schematic diagram of an integrating vector in accordance with another embodiment of the invention;

FIG. 4 is schematic diagram of a high copy episome vector in accordance with another embodiment of the invention;

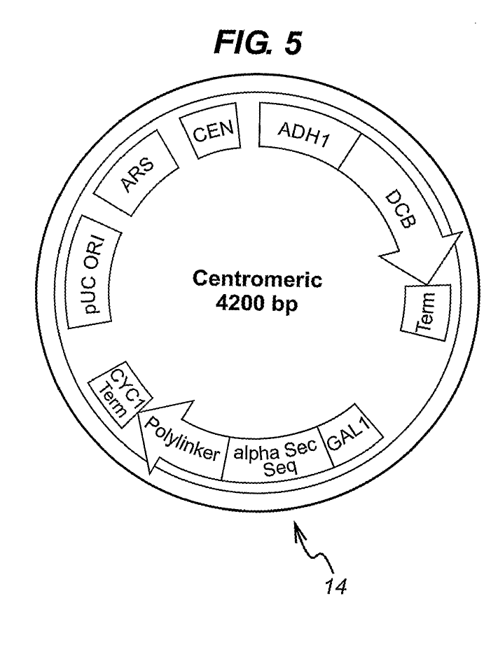

FIG. 5 is a schematic diagram of a centromeric vector in accordance with yet another embodiment of the invention;

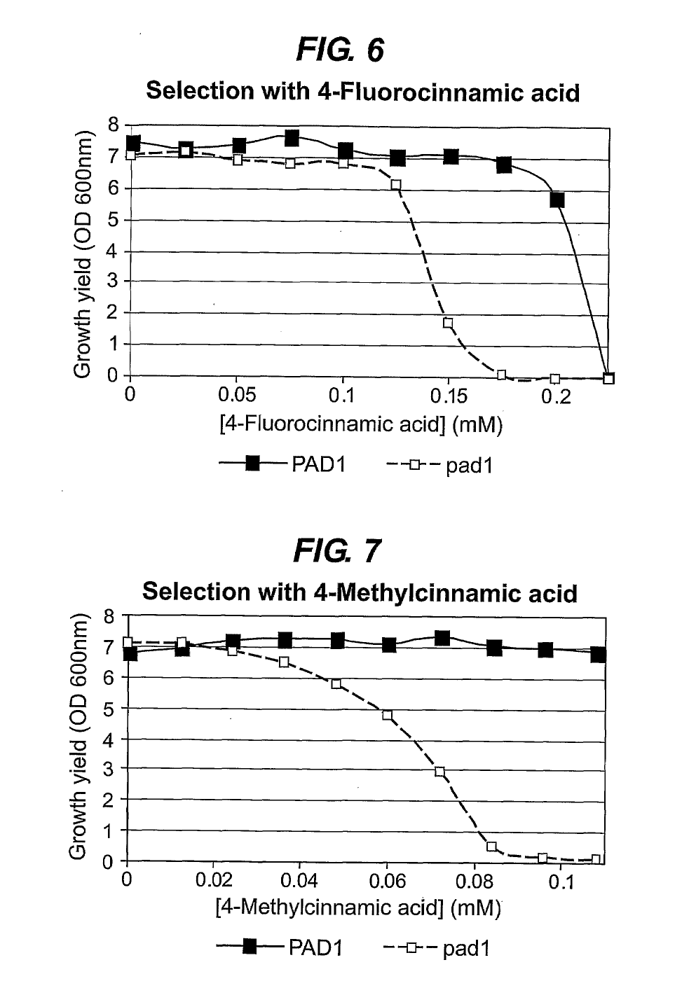

FIG. 6 is a graph showing the growth of yeast strains with (closed squares) and without (open squares) the PAD1 gene at a range of concentrations of the weak acid 4-fluorocinnamic acid;

FIG. 7 is a graph showing the growth of yeast strains with (closed squares) and without (open squares) the PAD1 gene at a range of concentrations of the weak acid 4-methylcinnamic acid;

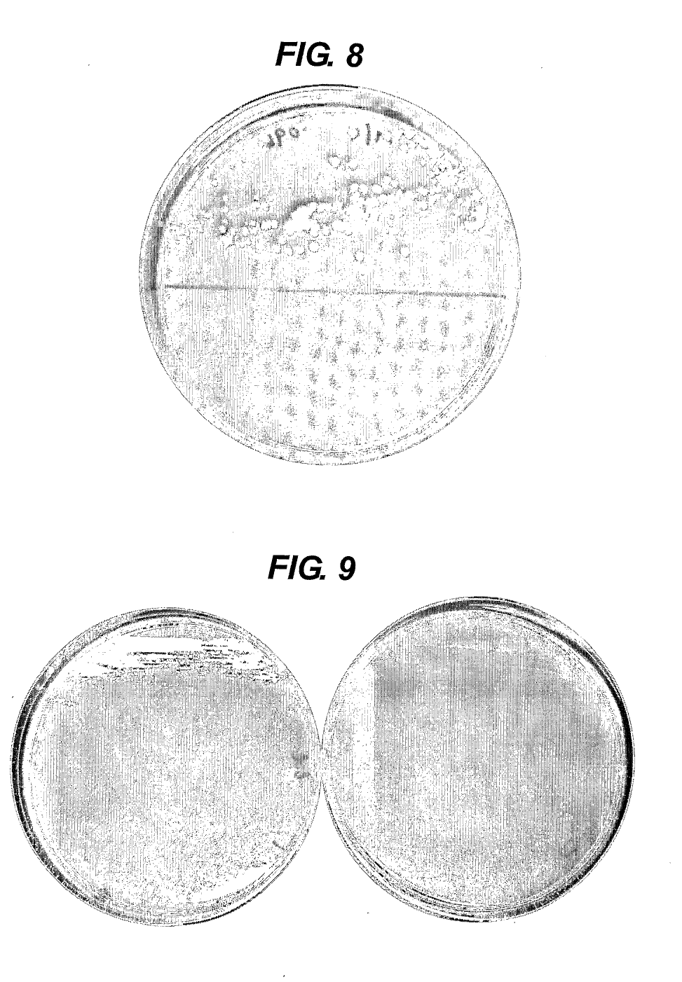

FIG. 8 is a photograph of an agar plate containing 4-methylcinnamic acid, plated with yeast with (top half) and without (bottom half) the PAD1 gene; and

FIG. 9 is a photograph of uracil-free agar, plated with yeast strain S. cerevisiae YO5833 (Δ pad1, ura3) electroporated with a plasmid containing PAD1 and a URA3 sequence (left plate) and without plasmid (right plate).

Reference is also made to the following sequence listings in which:

SEQ. ID NO: 1 is the open reading frame of the PAD1 gene of Saccharomyces cerevisiae; and

SEQ. ID NO: 2 is the protein sequence encoded by the gene sequence of SEQ. ID NO: 1.

The invention relates, in general terms, to the use of a nucleic acid encoding a decarboxylation enzyme as a selectable marker in a recombinant micro organism.

There is no structural feature that it is essential that the decarboxylation enzyme (hereinafter referred to as a “decarboxylase”) must have since different decarboxylases are known to have different structures. The common feature of decarboxylases is, therefore, the functional effect of being able to remove the carboxyl group of a weak acid. An exemplary scheme where sorbic acid is decarboxylated is shown in FIG. 1. More specifically, the COOH group of sorbic acid is cleaved off by the decarboxylase enzyme to form 1,3 pentadiene.

One test for a decarboxylase is described in A. Plumridge, S. J. A. Hesse, A. J. Watson, Lowe K. C., Strafford M and Archer D. B. (2004). The weak acid sorbic acid inhibits conidial germination and mycelial growth of Aspergillus niger through intracellular acidification. Applied and Environmental Microbiology vol. 70, 3506-3511. In this test, the presence of volatile hydrocarbons formed by decarboxylation is detected using GCMS of the headspace. Plumridge et al disclose a test in which the decarboxylase from mould spores completely removed 1 mM sorbic acid, and an equimolar amount of 1,3-pentadiene was found in the headspace by GCMS within 18 hours. The same assay can be used to test other potential decarboxylases. In some tests, the conversion of greater than 50% of the weak acid, as measured by the presence of the volatile hydrocarbon, after seven days incubation at 25 to 30° C. is in indication that the enzyme in question is a decarboxylase within the meaning of the present invention.

In preferred embodiments, the decarboxylase is at least 48.6% identical to the phenylacrylic acid decarboxylase enzyme (PAD) which is provided in SEQ. ID NO. 2. 48.6% identity is the identity to the product of the dedF of Escherichia coli (Clausen et al). In some embodiments, the decarboxylase is at least 32% identical to PAD in SEQ. ID NO. 2, this being the level of identity of the most distant homologue identified in Table 4. In still further embodiments, the decarboxylase is at least 30%, 40%, 50%, 60%, 70%, 80%, 90% or most preferably 100% identical to the PAD polypeptide of SEQ. ID NO. 2.

An example of a decarboxylase which is not structurally related to PAD but which can nevertheless be used in the present invention is pyruvate decarboxylase from Saccharomyces cerevisiae.

Where reference is made in the specification to a level of “identity” between two sequences then algorithms known in the art may be used to determine the level of identity.

For example the percentage identity between two sequences can be determined using the BLASTP algorithm version 2.2.2 (Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters.

However, it must be noted that structural homology to PAD is not essential to the invention.

The micro organism used in the present invention may be any micro organism which is a prokaryote or lower eukaryote (that is to say, a single celled eukaryote or a filamentous fungus). It is particularly preferred that the micro organism be a yeast cell such as Saccharomyces cerevisiae.

In some embodiments of the present invention, the nucleic acid sequence encoding the decarboxylase is part of a vector such as a plasmid. It is preferred that the vector comprises, in addition, a number of further elements to facilitate its use.

Referring to FIG. 2, an exemplary plasmid vector 1 is shown. The plasmid 1 comprises the PAD1 gene 2, which is SEQ. ID NO: 1, and, upstream thereof, a prokaryotic promoter 3. This permits the PAD1 gene 2 to be expressed in bacteria. Further upstream of the prokaryotic promoter 3 is a yeast promoter 4 which permits expression of the PAD1 gene 2 in yeast cells. It is preferred that the yeast promoter be a constitutive promoter providing at least three times higher expression of the PAD1 gene than is found in the endogenous gene.

In some embodiments, this additional promoter 11 is constitutive but it is preferably an inducible promoter. Suitable inducible promoters include GAL1, GAL7 and GAL10. Other suitable promoters are also disclosed in WO2004/042036. It is to be appreciated, however, that the prokaryotic promoter 3 and the yeast promoter 4 must not be the same promoter as the additional promoter 11 because the promoters might otherwise undergo homologous recombination.

In preferred embodiments, a terminator sequence (not shown) is provided downstream of the exogenous nucleic acid sequence 6. The presence of a terminator sequence greatly increases yields of the exogenous nucleic acid sequence 6. A preferred terminator is CYC1. Other exemplary terminators include TRP1, ADH1, GAP and MFI (Romanos et al).

The plasmid vector 1 also comprises a multiple cloning site 5 to permit insertion of an exogenous nucleic acid sequence 6 that is to be introduced into a host cell. Upstream of the multiple cloning site 5 is a secretion sequence 7 for example, the alpha factor sequence. The secretion sequence 7 causes the exogenous nucleic acid sequence to be secreted out of a cell in which it is expressed.

Upstream of the secretion sequence 7 is provided an additional promoter which is capable of effecting expression of the exogenous nucleic acid sequence 6.

The plasmid vector 1 also comprises a prokaryotic origin of replication 8 such as, for example, the E. coli OR1 origin of replication.

The plasmid vector 1 also comprises an autonomous replication sequence 9 (ARS). This permits a high number of copies of the plasmid 1 to exist and be replicated on cell division in a yeast cell. In some alternative embodiments, a Centromere (CEN) sequence is also present in the plasmid which results in more stable replication of the plasmid vector but in lower copy number. In either type of embodiment, the plasmid 1 remains independent of the yeast genome.

In some alternative embodiments, neither an ARS sequence 9 nor a CEN sequence is provided and instead a homologous recombination sequence 10 (such as, for example, the 26S ribosomal subunit spacer) is provided in the plasmid vector 1. When the plasmid vector 1 is introduced into a host cell, the homologous recombination sequence 10 results in heterologous recombination of the plasmid vector 1 into the host cell genome. Although FIG. 2 shows the vector 1 with both an ARS sequence 9 and a homologous recombination sequence 10, this is for reference purposes only and a vector 1 would typically have only one such sequence or the other.

In order to use the plasmid vector 1, an exogenous nucleic acid sequence 6 is inserted into the plasmid 1 at the multiple cloning sites using standard molecular biology protocols. It is to be appreciated that this is carried out on a large number of plasmids simultaneously. A sample of host cells is then transfected with the plasmids 1, again using techniques known in the art. The resulting host cells contain mostly untransformed cells mixed with a small number of transformed cells. The mixture is plated out on agar comprising a weak acid.

Referring to FIGS. 3, 4 and 5, three more specific embodiments of the present invention are shown. The following is an explanation of the terminology which is used in these figures.

“ADHI” is a promoter sequence derived from yeast Saccharomyces cerevisiae. This is a strong constitutive promoter, active in both yeast and bacteria.

“DCB” is the open reading frame of the DeCarBoxylase enzyme. This is expressed strongly in yeast and bacteria and is the selection resistance marker, conferring resistance to weak acids in both yeasts and bacteria. The DCB gene may originate from yeast, mould, bacterial, or animal or plant material, PAD1 is an example of a DCB.

“Term” is a termination sequence. A termination sequence may be beneficial to ensure efficient termination of the DCB transcript. Possible terminators include the termination sequence of TRP1, ADH1, GAP or MFI.

“GAL1” is an inducible promoter derived from yeast. This is strongly repressed by glucose, but expressed in the presence of galactose.

“alpha Sec Seq” is the alpha factor secretion signal sequence from Saccharomyces cerevisiae. This ensures secretion of the transcribed protein out from the cell and into the media around the cell.

“Polylinker” is a Multiple cloning site which is a series of unique cutting sites for restriction endonucleases. This is designed for efficient and easy insertion of the open reading frame of any heterologous protein needing expression in yeast.

“CYC-term” is an efficient termination sequence, derived from the CYC1 gene of Saccharomyces cerevisiae. This is to ensure efficient termination of the heterologous protein ORF. (It is to be noted that it is essential to avoid using identical termination sequences or promoters, in both the heterologous protein and in the DCB expression cassette).

“pUC ORI” is a bacterial origin of replication. It is present to effect efficient replication of the plasmid in bacteria.

“ARS” is a yeast replication sequence. It is present to effect efficient replication of the plasmid in yeast.

“CEN” is a centromeric sequence for attachment of the plasmid to a microtubule spindle.

“rDNA” is a long sequence of ribosomal DNA from either the 18S or 26S rDNA. rDNA sequences are found in all yeasts and are highly conserved. Different yeast species vary by <1%. The plasmid localises the homologous sequence in the yeast chromosomes and integrates into this site, without impairing yeast growth. Some yeast species contain multiple rDNA sequences, thus potentially enabling multiple integration of the plasmid.

Referring to FIG. 3, an integrating vector 12 comprises a gene encoding a decarboxylase enzyme, with a termination sequence downstream thereof and the ADH1 promoter sequence upstream thereof. It is also provided with the pUC ORI bacterial origin of replication. Furthermore, it is provided with a polylinker sequence with the termination sequence derived from the CYC1 gene thereof and the alpha factor secretion signal sequence upstream thereof. The GALL inducible promoter is upstream of the alpha factor secretion signal sequence.

The vector 12 is also provided with the pUC OR1 bacterial origin of replication and an rDNA sequence.

The plasmid 12 is capable of growing in yeast cells, in lower copy number, and integrating into the chromosomes of the host yeast cell via the rDNA sequence. The plasmid 12 is also capable of growing in bacteria such as E. coli as an episomal plasmid. A method of using an rDNA sequence for integration of a plasmid is described by Mackenzie D. A., Wongwathanarat P., Carter A. T. and Archer D. B. (2000) Isolation and use of a homologous histone H4 promoter and a ribosomal DNA region in a transformation vector for the oil-producing fungus Mortierella alpine. Applied and Environmental Microbiology vol. 66, 4655-4661.

Referring to FIG. 4, a plasmid vector 13 comprises the same elements as the plasmid vector 12 except that the rDNA sequence is replaced with a yeast origin of replication. The plasmid vector 13 is capable of growing in yeast cells as a high copy number episomal plasmid and is also capable of growing in bacteria such as E. coli as an episomal plasmid.

Referring to FIG. 5, a plasmid vector 14 comprises the same elements as the plasmid vector 13 except that it additionally comprises a centromeric sequence. The plasmid vector 14 is capable of growing in yeast cells as a low copy number episomal plasmid and also to grow in bacteria such as E. coli. Effectively, the centromeric plasmid vector 14 acts as a small extra chromosome.

In order to provide selection for host cells expressing a decarboxylase enzyme, a weak acid is provided to a solution of the cells before, for example plating the cells out on agar, or the weak acid may be incorporated in the agar growth medium.

In this specification, a weak acid is defined as an acid having a pKa ranging between pH 2.5 and pH 5.5. It is preferred that the weak acid has a pKa of between pH 4 and pH5. Exemplary weak acids are provided in Tables 1 and 2.

It is preferred that the host cells are exposed to the weak acids at a pH of less than 4.5. The reason for this is that the acid is in dynamic equilibrium between its acid form (HA) and its conjugate base form (H⁺A⁻) and it can only pass through the plasma membrane of a cell in the acid (HA) form. Therefore, by providing the weak acid at a relatively low pH, the equilibrium is driven to the acid form and more of the acid is able to pass inside the plasma membrane of the cell where it can be effective.

If the host cell is a eukaryote, then it is particularly preferred to provide the weak acid at a pH of 4 or lower because bacteria are unable to grow at such a low pH and so cannot infect the growth medium. It is therefore not necessary to sterilise the equipment in such embodiments because the low pH, itself, prevents any bacterial growth.

The presence of the weak acid inhibits cells which have not been transformed with the decarboxylase. The weak acid will normally kill such cells, but, in some embodiments, it may simply prevent growth and/or division of the untransformed cell. Cells which have been transformed with the recombinant decarboxylase, on the other hand, express the gene at high levels and the decarboxylase enzyme inactivates the weak acid by cleaving the COOH group from the acid. Thus the transformed cells are able to survive and replicate even in relatively high concentrations of weak acid (up to, for example, 10 mM). The process therefore ensures that the mixture of cells is enriched with transformed cells.

In embodiments of the invention which utilise PAD1 (or a structurally similar enzyme) as the decarboxylase enzyme, it is preferred that the weak acid is an aromatic weak acid such as coumaric acid, cinnamic acid or ferulic acid.

In preferred embodiments, the weak acid is a monocarboxylic acid because monocarboxylic acids are sufficiently toxic to micro organisms such as prokaryotes and yeasts, to be used as a selective medium Furthermore, monocarboxylic acids are recognised and decarboxylated by PAD1,

In some embodiments, instead of providing the weak acid, itself, an addition of the salt of the acid is provided. Sodium and potassium salts are particularly preferred. For example, in one embodiment potassium sorbate is added and is dissolved in a solution containing the host cells after which, the pH of the solution is lowered so that sorbic acid is formed by ressociation. Similarly sodium benzoate may be added to the host cell and benzoic acid is formed by ressociation. Indeed most weak acids can be provided in this way.

Although the above embodiments have described a plasmid vector, it is to be understood that other vectors may also be used. Thus the term “vector” includes plasmids, cosmids, viruses and phage.

There are several advantages to the present invention over the selectable markers known in the art. Weak acids are readily available and easily handled compared with antibiotics Letizia C. S., Cocchiara J., Lapczynski A., Lalko J and Api A. M. (2005) Fragrance material review on cinnamic acid. Food and Chemical Toxicology 43, 925-943. Weak acids are not, for example, heat-sensitive as many antibiotics are and so it is possible to add the weak acid to a nutrient broth before heat sterilisation of the broth. Furthermore, weak acids are not used in the medical field and therefore the existence of micro organisms with resistance to weak acids is not a medical concern. In addition, the selectable marker can be used even in fungal host cells, such as yeast. It is also possible to use the selectable markers of the present invention in wild-type yeast host cells, auxotrophic mutants or strains not being necessary.

TABLE 1
(158 Monocarboxylic Weak Acids)

| Weak Acid | CAS Number | Pka |
|---|---|---|
| Formic acid | 64-18-6 | 3.75 |
| Acetic acid | 64-19-7 | 4.76 |
| Chloroacetic acid | 79-11-8 | 2.87 |
| Propionic acid | 79-09-4 | 4.81 |
| Acrylic acid = 2-propenoic acid | 79-10-7 | 4.25 |
| Methacrylic acid = 2-methyl-2-propenoic | 79-41-4 | 4.61 |
| acid | | |
| 3,3-Dimethylacrylic acid | 541-47-9 | 5.0806 |
| Propiolic acid = 2-propynoic acid | 471-25-0 | 2.39 |
| Isobutyric acid = 2-Methylpropionic acid | 79-31-2 | 4.84 |
| Trimethylacetic acid = 3,3- | 75-98-9 | 5.03 |
| Dimethylpropionic acid | | |
| 2-Chloropropionic acid | 598-78-7 | 2.897 |
| 3-Chloropropionic acid | 107-94-8 | 3.8924 |
| 2-Chloroacrylic acid | 598-79-8 | 2.6975 |
| trans-3-Chloroacrylic acid | 2345-61-1 | 3.2506 |
| cis-3-Chloroacrylic acid | 1609-93-4 | 3.2506 |
| 2,2-Dichloropropionic acid | 75-99-0 | |
| Butyric acid | 107-92-6 | 4.82 |
| Crotonic acid = 2-Butenoic acid | 107-93-7 | 4.17 |
| Vinylacetic acid = 3-Butenoic acid | 625-38-7 | 4.51 |
| Tiglic acid | 80-59-1 | 4.4564 |
| 2-Butynoic acid | 590-93-2 | 2.6546 |
| 2-Methylbutyric acid | 116-53-0 | 4.81 |
| Isovaleric acid = 3-Methylbutyric acid | 503-74-2 | 4.77 |
| 2,2-Dimethylbutyric acid | 595-37-9 | 5.03 |
| 3,3-Dimethylbutyric acid | 1070-83-3 | 4.961 |
| 2-Ethylbutyric acid | 88-09-5 | 4.71 |
| Valeric acid | 109-52-4 | 4.84 |
| trans-2-Pentenoic acid | 13991-37-2 | 4.1578 |
| trans-3-Pentenoic acid | 5204-64-8 | 4.36 |
| 4-Pentenoic acid | 591-80-0 | 4.6364 |
| 4-Pentynoic acid | ″6089-09-4 | 4.1342 |
| 2-Methylvaleric acid | 97-61-0 | 4.79 |
| 3-Methylvaleric acid | 105-43-1 | 4.7868 |
| 4-Methylvaleric acid | 646-07-1 | 4.8689 |
| 2,4-Pentadienoic acid | 626-99-3 | 4.72 |
| Hexanoic acid | 142-62-1 | 4.88 |
| trans-2-Hexenoic acid | 13419-69-7 | 3.8306 |
| trans-3-Hexenoic acid | 1577-18-0 | 4.608 |
| cis-3-Hexenoic acid | 4219-24-3 | 4.608 |
| trans-4-Hexenoic acid | 35194-36-6 | 4.6984 |
| 5-Hexenoic acid | 1577-22-6 | 4.81 |
| Sorbic acid = 2,4-hexadienoic acid | 110-44-1 | 4.76 |
| 5-Hexynoic acid | 53293-00-8 | 4.5589 |
| Heptanoic acid | 111-14-8 | 4.8 |
| trans-2-Heptenoic acid | 10352-88-2 | 3.7839 |
| 3-Heptenoic acid | | 4.7696 |
| 6-Heptenoic acid | 1119-60-4 | 4.81 |
| trans, trans-2,6-Heptadienoic acid | 38867-17-3 | 3.7528 |
| 6-Heptynoic acid | 30964-00-2 | 4.70956 |
| 2,4-Heptadienoic acid | | |
| Octanoic acid | 124-07-2 | 4.89 |
| trans-2-Octenoic acid | 1871-67-6 | 3.90184 |
| 3-Octenoic acid | 1577-19-1 | 4.7926 |
| 2,4-Octadienoic acid | | |
| Nonanoic acid | 112-05-0 | 4.95 |
| 2-Nonenoic acid | 3760-11-0 | 3.9148 |
| 2,4-Nonadienoic acid | | |
| Decanoic acid | 334-48-5 | 4.9 |
| 2-Decenoic acid | 334-49-6 | 3.9018 |
| 3-Decenoic acid | 53678-20-9 | 4.728 |
| 2,4-Decadienoic acid | | |
| Undecanoic acid | 112-37-8 | 5.03 |
| (R)(+)Citronellic acid | 18951-85-4 | 4.75185 |
| Geranic acid | 459-80-3 | 4.7211 |
| Benzoic acid | 65-85-0 | 4.19 |
| o-Toluic acid = 2-Methylbenzoic acid | 118-90-1 | 3.98 |
| m-Toluic acid = 3-Methylbenzoic acid | 99-04-7 | 4.27 |
| p-Toluic acid = 4-Methylbenzoic acid | 99-94-5 | 4.37 |
| 2-Picolinic acid = Pyridine-2-carboxylic acid | 98-98-6 | 5.39 |
| Salicylic acid = 2-Hydroxybenzoic acid | 69-72-7 | 2.97 |
| p-Hydroxybenzoic acid = 4-hydroxybenzoic | 99-96-7 | 4.54 |
| acid | | |
| 2-Chlorobenzoic acid | 118-91-2 | 2.89 |
| 3-Chlorobenzoic acid | 535-80-8 | 3.81 |
| 4-Chlorobenzoic acid | 74-11-3 | 3.96 |
| 2-Nitrobenzoic acid | 552-16-9 | 2.47 |
| 3-Nitrobenzoic acid | 121-92-6 | 3.46 |
| 4-Nitrobenzoic acid | 62-23-7 | 3.42 |
| 2-Aminobenzoic acid = Anthranillic acid | | |
| 3-Aminobenzoic acid | | |
| 4-Aminobenzoic acid | | |
| Vanillic acid | 121-34-6 | 4.51 |
| m-Anisic acid | 586-38-9 | 4.09 |
| 4-Ethylbenzoic acid | 619-64-7 | 4.35 |
| Coumaric acid | 501-98-4 | 4.64 |
| Cumic acid | 536-66-3 | 4.35 |
| 2-Hydroxy-cinnamic acid | 614-60-8 | 4.7 |
| 3-Hydroxy-cinnamic acid | 14755-02-3 | 4.6 |
| Caffeic acid | 331-39-5 | 4.7 |
| Ferulic acid | 1135-24-6 | 4.58 |
| Cinnamic acid | 140-10-3 | 4.44 |
| 2,3 or 4-Methyl-cinnamic acid | 2373-76-4; 14473-89-3; 1866-39-3 | 4.5 |
| alpha-methyl-cinnamic acid | 1199-77-5 | 4.45 |
| alpha Phenyl-cinnamic acid | 91-48-5 | |
| 2,3 or 4-Nitro-cinnamic acid | 621-41-9; 555-68-0; 619-89-6 | |
| 2,3, or 4-Fluoro-cinnamic acid | 451-69-4; 20595-30-6; 459-32-5 | 4.5 |
| alpha-fluoro-cinnamic acid | 350-90-3 | 4.45 |
| 2,3 or 4-Chloro-cinnamic acid | 3752-25-8; 1866-38-2; 1615-02-7 | 4.5 |
| 2,3 or 4-Bromo-cinnamic acid | 7345-79-1; 32862-97-8; 1200-07-3 | 4.5 |
| Difluoro-cinnamic acid, 2,3; 2,4; 2,5; 2,6; | 57014; 94977-52-3; 112898-33- | 4.5 |
| 3,4; or 3,5 | 6; 102082-89-3; 112897-97-9; | |
| | 147700-58-1 | |
| Dichloro-cinnamic acid, 2,3; 2,4; 2,5; 2,6; 3,4; | 20595-44-2; 20595-45-3; 1202- | 4.5 |
| or 3,5 | 39-7; 112898-33-6; 102082-89-3; | |
| | 147700-58-1 | |
| Dibromo-cinnamic acid | | |
| Trifluoro-cinnamic acid, 2,3,4; or 3,4,5 | 207742-85-6; 152152-19-7 | |
| 2,3,4,5,6-Pentafluoro-cinnamic acid | 719-60-8 | |
| 2,3 or 4-Iodo-cinnamic acid | | |
| 2,3 or 4-Ethyl-cinnamic acid | | |
| 2,3 or 4-Propyl-cinnamic acid | | |
| 2,3 or 4-Trifluoromethyl-cinnamic acid | 2062-25-1; 16642-92-5; 779-89-5 | 4.2 |
| 2,3, or 4-Trichloromethyl-cinnamic acid | | |
| 2,3 or 4-Methoxy-cinnamic acid | 6099-04-3; 6099-03-2; 830-09-1 | 4.5 |
| 2,3 or 4-Ethoxy-cinnamic acid | 69038-81-9; 103986-73-8; 2373- | 4.5 |
| | 79-7 | |
| Dimethoxy-cinnamic acid, 2,3; 2,4; 2,5; 2,6; | 7345-82-6; 16909-09-4; | 4.6 |
| 3,4; 3,5 | 10538 = 51-9; 2316-26-9; 16909- | |
| | 11-8; | |
| Trimethoxy-cinnamic acid, 2,3,4; or 3,4,5 | 90-50-6; 24160-53-0 | |
| Hydrocinnamic acid | 501-52-0 | 4.66 |
| trans-Styrylacetic acid | 1914-58-5 | 4.688 |
| 2-Carboxy-cinnamic acid | 612-40-8 | |
| 4-Formyl-cinnamic acid | 23359-08-2 | |
| 4-Dimethylamino-cinnamic acid | 1552-96-1 | |
| 4-Amino-cinnamic acid | 54057-95-3 | |
| 1,4-Phenylene-diacrylic acid | 16323-43-6 | |
| Phenyl pyruvic acid | | |
| Phenyl propiolic acid | 637-44-5 | |
| Cyclopropanecarboxylic acid | 1759-53-1 | 4.83 |
| 1,1-Cyclopropanedicarboxylic acid | 598-10-7 | 3.11417 |
| 1-Methyl-cyclopropane carboxylic acid | 6914-76-7 | 5.1 |
| 2-Methyl-cyclopropane carboxylic acid | 29555-02-0 | 4.8 |
| Cyclopropylacetic acid | 5239-82-7 | 4.8873 |
| Cyclobutanecarboxylic acid | 3721-95-7 | 4.8 |
| Cyclopentanecarboxylic acid | 3400-45-1 | 4.8 |
| 1-Cyclopentene-1-carboxylic acid | 1560-11-8 | 3.7528 |
| 1-Cyclopentene-acrylic acid | | |
| Cyclopentylacetic acid | 1123-00-8 | 4.76 |
| 2-Cyclopentene-1-acetic acid | 13668-61-6 | 4.60992 |
| 3-Cyclopentylpropionic acid | 140-77-2 | 4.81 |
| Thiophene-2-carboxylic acid | 527-72-0 | 3.53 |
| Thiophene-3-carboxylic acid | 88-13-1 | 4.08 |
| Cyclohexanecarboxylic acid | 98-89-5 | 4.9 |
| 1-Cyclohexene-1-carboxylic acid | 636-82-8 | 3.7528 |
| 1-Cyclohexene-acrylic acid | | |
| 3-Cyclohexene-1-carboxylic acid | 4771-80-6 | 4.59302 |
| Cyclohexylacetic acid | 5292-21-7 | 4.8806 |
| Cyclohexanepropionic acid | 701-97-3 | 4.8658 |
| Cyclohexanebutyric acid | 4441-63-8 | 4.9061 |
| 4-Phenylbutyric acid | 1821-12-1 | 4.76 |
| Cycloheptanecarboxylic acid | 1460-16-8 | 4.9518 |
| Cycloheptylacetic acid | 4401-20-1 | 4.82164 |
| 1-Cycloheptene-acrylic acid | | |
| 2-Furoic acid | 88-14-2 | 3.13 |
| Furylacrylic acid | 539-47-9 | 3.9424 |
| 3-Furan-acrylic acid | 81311-95-7 | 3.9 |
| 3-(2-Thienyl)acrylic acid | 15690-25-2 | 4.2 |
| 3-(3-Thienyl)acrylic acid | 102696-71-9 | 4.1 |
| Pyridinium acrylic acid | | |
| Pyrrolyl-acrylic acid | | |
| Phenylacetic acid | 103-82-2 | 4.31 |
| Phenoxyacetic acid | 122-59-8 | 3.17 |
| 2-Phenylpropionic acid | 492-37-5 | 4.0606 |
| 3-Indole-acrylic acid | 29953-71-7 | |
| 4-Imidazole-acrylic acid (Urocanic) | 104-98-3 | |

TABLE 2
(19 Most Preferred weak acids)

| Weak Acid | CAS Number | Pka |
|---|---|---|
| 2,4-Pentadienoic acid | 626-99-3 | 4.72 |
| Sorbic acid = 2,4-hexadienoic acid | 110-44-1 | 4.76 |
| Cinnamic acid | 140-10-3 | 4.44 |
| 2,3 or 4-Methyl-cinnamic acid | 2373-76-4; 14473-89-3; 1866-39-3 | 4.5 |
| alpha-methyl-cinnamic acid | 1199-77-5 | 4.45 |
| 2,3, or 4-Fluoro-cinnamic acid | 451-69-4; 20595-30-6; 459-32-5 | 4.5 |
| alpha-fluoro-cinnamic acid | 350-90-3 | 4.45 |
| 2,3 or 4-Chloro-cinnamic acid | 3752-25-8; 1866-38-2; 1615-02-7 | 4.5 |
| 2,3 or 4-Bromo-cinnamic acid | 7345-79-1; 32862-97-8; 1200-07-3 | 4.5 |
| Difluoro-cinnamic acid, 2,3; 2,4; 2,5; | 57014; 94977-52-3; 112898-33-6; 102082- | 4.5 |
| 2,6; 3,4; or 3,5 | 89-3; 112897-97-9; 147700-58-1 | |
| Dichloro-cinnamic acid, 2,3; 2,4; 2,5; | 20595-44-2; 20595-45-3; 1202-39-7; | 4.5 |
| 2,6; 3,4; or 3,5 | 112898-33-6; 102082-89-3; 147700-58-1 | |
| 2,3 or 4-Trifluoromethyl-cinnamic | 2062-25-1; 16642-92-5; 779-89-5 | 4.2 |
| acid | | |
| 2,3 or 4-Methoxy-cinnamic acid | 6099-04-3; 6099-03-2; 830-09-1 | 4.5 |
| 2,3 or 4-Ethoxy-cinnamic acid | 69038-81-9; 103986-73-8; 2373-79-7 | 4.5 |
| Dimethoxy-cinnamic acid, 2,3; 2,4; | 7345-82-6; 16909-09-4; 10538 = 51-9; 2316- | 4.6 |
| 2,5; 2,6; 3,4; 3,5 | 26-9; 16909-11-8; | |
| Furylacrylic acid | 539-47-9 | 3.9424 |
| 3-Furan-acrylic acid | 81311-95-7 | 3.9 |
| 3-(2-Thienyl)acrylic acid | 15690-25-2 | 4.2 |
| 3-(3-Thienyl)acrylic acid | 102696-71-9 | 4.1 |

### EXAMPLES

### Example 1

A range of weak acids were tested for their ability to be decarboxylated by yeast expressing a decarboxylation enzyme using the following protocol.

S. cerevisiae strain 62 (which expresses the PAD1 gene) was cultured for 48 hr in YEPD pH 4, Yeast extract 10 g, Peptone 20 g, Glucose 20 g, at 28° C. in a shake flask, 120 rpm. The stationary-phase cells were inoculated into capped 28 ml McCartney bottles containing 10 mls YEPD pH 4, at 1 million cells per ml. Each bottle also contained one of 70 substrate weak acids at 0.5 mM. Tubes were then shaken for 10 h at 28° C.

1 ml samples of the headspace were passed into stainless-steel thermal desorption tubes, followed by a 10 ml air flush. (Desorption tubes—89 mm×5 mm ID, packed with 200 mg Tenax TA 60-80 mesh, Phase Separations, UK).

Thermal desorption tubes were placed in a Perkin Elmer ATD400 thermal desorption system, and the volatiles transferred to the Gas Chromatograph in a 2 stage process. Primary desorption was 250° C. for 10 min with a helium flow of 401/min. Using a split ratio of 1:1, volatiles were retained on a cold trap (25 mg Tenax TA) held at −30° C. Secondary desorption was carried out by rapidly heating and holding the cold trap at 250° C. for 2 min, with a helium flow of 20 ml/min and split ratio of 19:1. A DB624 column {30 m×0.32 mm, 1.8 microns film thickness} installed in a Carbo Erba GC800 was used to achieve separation and introduction of the volatile to the mass spectrometer. The column temperature was held at 40° C. for 2 min then increased at 5° C./min to 250° C. The column outlet was coupled via a heated transfer line at 250° C. into the ion source of the mass spectrometer operated at 70 eV in electron ionization (EI) mode. The source temperature was at 200° C. and the detector photomultiplier at 500V. Full scan MS data acquisition was carried out from m/z 30 to 300, with 0.6 sec scan and 0.05 sec inter scan delay.

The results are shown in Table 3, in each case the “predicted product” being the product which would be produced if decarboxylation of the weak acid were to take place. The results clearly contrast those weak acids where no predicted product was detected (GCMS Peak Area=0) and thus decarboxylation did not occur and those weak acids where the predicted product was detected (GCMS Peak Area >0) and thus decarboxylation of the weak acid did occur.

The results for the decarboxylation of cinnamic acid may be confirmed using the method reported in Prim N., Pastor J. and Diaz P. (2002) Zymographic detection of cinnamic decarboxylase activity. J. Microbiol. Methods 51, 417-420. This method is based on a change in pH caused by cell-free extracts altering the pH as they consume cinnamic acid. An indicator dye, bromocresol purple, changes to yellow as the decarboxylase works.

TABLE 3
(Decarboxylation of weak acids by Saccharomyces cerevisiae strain 62
expressing PAD1)

| | | | GCMS |
|---|---|---|---|
| Weak acid | Predicted Product | MW | Peak area |
| Crotonic acid | 1-Propene | 42.080 | 0 |
| 2,4-Pentadienoic acid | 1,3-Butadiene | 54.1 | 107 |
| trans-2-Pentenoic acid | 1-Butene | 56.107 | 0 |
| Valeric acid | Butane | 58.123 | 0 |
| trans-2-Hexenoic acid | 1-Pentene | 70.134 | 0 |
| trans-3-Hexenoic acid | 2-Pentene | 70.134 | 0 |
| trans-4-Hexenoic acid | 3-Pentene | 70.134 | 0 |
| trans-5-Hexenoic acid | 4-Pentene | 70.134 | 0 |
| cis-3-Hexenoic acid | 2-Pentene | 70.134 | 0 |
| Sorbic acid | 1,3-Pentadiene | 68.102 | 8795 |
| Hexanoic acid | Pentane | 72.1 | 0 |
| Benzoic acid | Benzene | 78.000 | 0 |
| 2,6-Heptadienoic acid | 1,5-Hexadiene | 82.1 | 0 |
| trans-2-Heptenoic acid | 1-Hexene | 84.161 | 0 |
| Heptanoic acid | Hexane | 86.2 | 0 |
| Furylacrylic acid | 2-Vinyl-furan | 94.1 | 15611 |
| 3-Furan-acrylic acid | 3-Vinyl-furan | 94.1 | 2165 |
| Urocanic acid | 4-Vinyl-1H-imidazole | 94.116 | 0 |
| trans-2-Octenoic acid | 1-Heptene | 98.188 | 0 |
| Phenyl-propiolic acid | Ethynyl-benzene | 102.135 | 0 |
| Cinnamic acid | Styrene | 104.2 | 17538 |
| Cinnamyl alcohol | Styrene | 104.151 | 0 |
| Cinnamaldehyde | Styrene | 104.151 | 0 |
| Hydrocinnamic acid | Ethyl-benzene | 106.000 | 0 |
| 3-(3-Thienyl)acrylic acid | 3-Vinyl-thiophene | 110.2 | 1586 |
| 3-(2-Thienyl)acrylic acid | 2-Vinyl-thiophene | 110.2 | 20571 |
| trans-2-Nonenoic acid | 1-Octene | 112.214 | 0 |
| 4-Methyl-cinnamic acid | 4-Methyl-styrene | 118.2 | 11013 |
| alpha-methyl-cinnamic acid | Propenyl-benzene | 118.2 | 889 |
| 2-Methyl-cinnamic acid | 2-Methyl-styrene | 118.2 | 18046 |
| 3-Methyl-cinnamic acid | 3-Methyl-styrene | 118.2 | 8195 |
| trans-Styrylacetic acid | 2-Propenyl-benzene | 118.178 | 0 |
| 2-Hydroxy-cinnamic acid | 2-Hydroxy-styrene | 120.2 | 0 |
| 4-Hydroxy-cinnamic acid | 4-Hydroxy-styrene | 120.151 | 0 |
| 3-Hydroxy-cinnamic acid | 3-Hydroxy-styrene | 120.151 | 0 |
| alpha fluorocinnamic acid | alpha fluorostyrene | 122.0 | 2659 |
| 2-Fluoro-cinnamic acid | 2-Fluoro-styrene | 122.1 | 3760 |
| 3-Fluoro-cinnamic acid | 3-Fluoro-styrene | 122.1 | 14027 |
| 4-Fluoro-cinnamic acid | 4-Fluoro-styrene | 122.1 | 18699 |
| Geranic acid | 2,6-Dimethyl-hepta-1,5- | 124.2 | 0 |
| | diene | | |
| trans-2-Decenoic acid | 1-Nonene | 126.241 | 0 |
| 3-[4-(2-Carboxy-vinyl)-phenyl]-acrylic | 1,4-Divinyl-benzene | 130.2 | 0 |
| acid | | | |
| 4-Formyl-cinnamic acid | 4-Formyl-styrene | 132.162 | 0 |
| 2-Methoxy-cinnamic acid | 2-Methoxy-styrene | 134.2 | 128 |
| 3-Methoxy-cinnamic acid | 3-Methoxy-styrene | 134.2 | 178 |
| 4-Methoxy-cinnamic acid | 4-Methoxy-styrene | 134.2 | 1899 |
| 3,4-Dihydroxy-cinnamic acid | 3,4-Dihydroxy-styrene | 136.150 | 0 |
| 2-Chloro-cinnamic acid | 2-Chloro-styrene | 138.0 | 4911 |
| 3-Choro-cinnamic acid | 3-Chloro-styrene | 138.6 | 907 |
| 4-Choro-cinnamic acid | 4-Chloro-styrene | 138.6 | 12391 |
| 2,4-Difluoro-cinnamic acid | 2,4-Difluoro-styrene | 140.1 | 35698 |
| Indole-acrylic acid | 3-Vinyl-1H-indole | 143.188 | 0 |
| 4-Dimethylamino-cinnamic acid | 4-Dimethylamino-styrene | 147.000 | 0 |
| 2-Ethoxycinnamic acid | 2-ethoxystyrene | 148.0 | 193 |
| 4-Ethoxycinnamic acid | 4-ethoxystyrene | 148.0 | 227 |
| 2-Carboxy-cinnamic acid | 2-Vinyl-benzoic acid | 148.161 | 0 |
| 2-Nitro-cinnamic acid | 2-Nitro-styrene | 149.1 | 0 |
| 4-Nitro-cinnamic acid | 4-Nitro-styrene | 149.149 | 0 |
| 3-Nitro-cinnamic acid | 3-Nitrostyrene | 149.149 | 0 |
| 4-Hydroxy, 3-methoxy-cinnamic acid | 4-Hydroxy-3-methoxy- | 150.177 | 0 |
| | styrene | | |
| 2,3,4-Trifluoro-cinnamic acid | 2,3,4-trifluorostyrene | 158.000 | 0 |
| 2,4-Dimethoxy-cinnamic acid | 2,4-Dimethoxy-styrene | 164.2 | 30 |
| 3-Trifluoromethyl-cinnamic acid | 3-Trifluoromethyl-styrene | 172.1 | 946 |
| 2-Trifluoromethyl-cinnamic acid | 2-Trifluoromethyl-styrene | 172.1 | 1440 |
| 4-Trifluoromethyl-cinnamic acid | 4-Trifluoromethyl-styrene | 172.1 | 1600 |
| trans-2,4-Dichloro-cinnamic acid | 2,4-Dichloro-styrene | 173.0 | 4428 |
| alpha-phenyl-cinnamic acid | 3-Styryl-benzene | 180.249 | 0 |
| 4-Bromo-cinnamic acid | 4-Bromo-styrene | 183.0 | 1038 |
| 2-Bromo-cinnamic acid | 2-Bromo-styrene | 183.0 | 2912 |
| 3-Bromo-cinnamic acid | 3-Bromo-styrene | 183.0 | 248 |
| 2,3,4-Trimethoxy-cinnamic acid | 2,3,4-Trimethoxy-styrene | 194.230 | 0 |

### Example 2

A search of the EMBL protein database (www.ebi.ac.uk) was carried out for Saccharomyces cerevisiae Pad1p protein homologues using the WU-Blast2 algorithm. The results are shown in Table 4. The results provide a list of other decarboxylation enzymes which may be used in the present invention. The results also indicate the level of structural identity to the Pad1p protein that is necessary for decarboxylase activity to be retained.

TABLE 4
(Saccharomyces cerevisiae Pad1p protein homologues)

| | Accession | | | | |
|---|---|---|---|---|---|
| Organism | number | Protein description | Length | Identity % | E value |
| Saccharomyces | P33751 | Phenylacrylic acid | 242 | 100 | 6.9e−125 |
| cerevisiae | | decarboxylase (EC 4.1.1.—) | | | |
| | | (PAD). | | | |
| Debaryomyces hansenii | Q6BJQ7 | Similar to sp\|P33751 | 284 | 73 | 8.2e−74 |
| | | Saccharomyces cerevisiae | | | |
| | | Phenylacrylic acid | | | |
| | | decarboxylase. | | | |
| Candida albicans | Q5A8L8 | Hypothetical protein PAD1. | 229 | 72 | 2.6e−70 |
| Gibberella zeae | Q4I8M3 | Hypothetical protein. | 225 | 62 | 4.3e−61 |
| Nectria haematococca | Q873Q9 | Putaive phenyacrylic acid | 169 | 60 | 7.5e−48 |
| | | decarboxylase (Fragment). | | | |
| Nectria haematococca | Q8J0Q6 | Hypothetical protein | 168 | 60 | 9.6e−48 |
| | | (Fragment). | | | |
| Aspergillus nidulans | Q5AX17 | Hypothetical protein. | 739 | 58 | 1.1e−32 |
| Bacillus subtilis | P94404 | Probable aromatic acid | 204 | 57 | 3.4e−52 |
| | | decarboxylase (EC 4.1.1.—). | | | |
| Bacillus licheniformis | Q65NI2 | YclB (3-octaprenyl-4- | 189 | 56 | 9.0e−52 |
| | | hydroxybenzoate carboxylyase). | | | |
| Enterobacter cloacae | Q8VNT1 | Putative Pad1. | 200 | 55 | 6.4e−51 |
| Salmonella paratyphi | Q5PEF3 | Hypothetical protein. | 673 | 54 | 3.5e−50 |
| Escherichia coli | Q9F8R0 | Phenylacrylic acid | 197 | 54 | 7.3e−50 |
| | | decarboxylase-like protein. | | | |
| Moorella thermoacetica | Q3XFJ6 | Phenylacrylic acid | 188 | 54 | 9.3e−50 |
| | | decarboxylase. | | | |
| Escherichia coli | P69772 | Probable aromatic acid | 197 | 54 | 5.1e−49 |
| | | decarboxylase (EC 4.1.1.—). | | | |
| Escherichia coli | P69774 | Probable aromatic acid | 197 | 54 | 5.1e−49 |
| | | decarboxylase (EC 4.1.1.—). | | | |
| Shigella flexneri | Q83QE5 | Phenylacrylic acid | 197 | 54 | 5.1e−49 |
| | | decarboxylase-like protein. | | | |
| Mycobacterium | Q73ZG8 | Hypothetical protein. | 207 | 53 | 1.4e−48 |
| paratuberculosis | | | | | |
| Escherichia coli | P69773 | Probable aromatic acid | 197 | 53 | 4.6e−48 |
| | | decarboxylase (EC 4.1.1.—). | | | |
| Geobacillus kaustophilus | Q5QL33 | Phenylacrylic acid | 180 | 53 | 5.9e−48 |
| | | decarboxylase. | | | |
| Kluyvera citrophila | Q8VN19 | Putative Pad1 protein. | 196 | 52 | 1.6e−47 |
| Methanosarcina | Q8TST4 | Phenylacrylic acid | 183 | 52 | 4.3e−45 |
| acetivorans | | decarboxylase. | | | |
| Rickettsia prowazekii | Q9ZD09 | Probable aromatic acid | 189 | 51 | 3.0e−44 |
| | | decarboxylase (EC 4.1.1.—). | | | |
| Pseudomonas | Q3KAN5 | Phenylacrylic acid | 207 | 51 | 7.2e−43 |
| fluorescens | | decarboxylase. | | | |
| Wolbachia endosymbiont | Q4E7X3 | Phenylacrylic acid | 191 | 50 | 6.3e−44 |
| of Drosophila simulans | | decarboxylase, 3-octaprenyl- | | | |
| | | 4-hydroxybenzoate carboxylyase | | | |
| | | (EC 4.1.1.—). | | | |
| Wolbachia pipientis | Q73HK2 | Phenylacrylic acid | 191 | 50 | 8.0e−44 |
| | | decarboxylase, 3-octaprenyl- | | | |
| | | 4-hydroxybenzoate carboxylyase | | | |
| | | (EC 4.1.1.—). | | | |
| Methanosarcina barkeri | Q46C19 | Phenylacrylic acid | 183 | 50 | 1.0e−43 |
| | | decarboxylase. | | | |
| Wolbachia endosymbiont | Q5D5E5 | Phenylacrylic acid | 186 | 50 | 2.4e−35 |
| of Drosophila mojavensis | | decarboxylase, 3-octaprenyl- | | | |
| | | 4-hydroxybenzoate carboxylyase | | | |
| | | (EC 4.1.1.—). | | | |
| Ralstonia eutropha | Q46WP6 | Phenylacrylic acid | 196 | 49 | 3.4e−45 |
| | | decarboxylase. | | | |
| Ralstonia eutropha | Q46WS5 | Phenylacrylic acid | 208 | 49 | 4.3e−45 |
| | | decarboxylase. | | | |
| Archaeoglobus fulgidus | O29054 | Probable aromatic acid | 182 | 49 | 1.3e−41 |
| | | decarboxylase (EC 4.1.1.—). | | | |
| Shewanella oneidensis | Q8EAT8 | Phenylacrylic acid | 219 | 49 | 4.6e−27 |
| | | decarboxylase, 3-octaprenyl- | | | |
| | | 4-hydroxybenzoate carboxylyase, | | | |
| | | putative. | | | |
| Burkholderia | Q63QE7 | Putative aromatic acid | 198 | 48 | 2.4e−44 |
| pseudomallei | | decarboxylase. | | | |
| Thauera aromatica | P57767 | Probable aromatic acid | 194 | 48 | 7.2e−43 |
| | | decarboxylase (EC 4.1.1.—). | | | |
| Agrobacterium | Q8U7Q6 | Phenylacrylic acid | 196 | 48 | 6.7e−40 |
| tumefaciens | | decarboxylase | | | |
| | | (AGR_L_942p). | | | |
| Methanococcoides | Q41PH4 | Phenylacrylic acid | 183 | 47 | 3.1e−42 |
| burtonii | | decarboxylase. | | | |
| Sphingomonas | O85998 | Decarboxylase precursor. | 204 | 47 | 1.3e−41 |
| aromaticivorans | | | | | |
| Pyrococcus horikoshii | O58742 | Probable aromatic acid | 181 | 47 | 9.8e−39 |
| | | decarboxylase (EC 4.1.1.—). | | | |
| Rhizobium meliloti | Q92XP7 | Probable decarboxylase. | 197 | 46 | 4.6e−41 |
| Rhizobium meliloti | Q92Z12 | Probable decarboxylase | 200 | 46 | 4.7e−39 |
| | | (Hypthetical protein). | | | |
| Pyrococcus abyssi | Q9V030 | Probable aromatic acid | 181 | 46 | 6.9e−38 |
| | | decarboxylase (EC 4.1.1.—). | | | |
| Pseudoalteromonas | Q3IHV3 | Putative aromatic acid | 174 | 46 | 1.2e−22 |
| haloplanktis | | decarboxylase (EC 4.1.1.—). | | | |
| Pyrobaculum aerophilum | Q8ZX39 | Phenylacrylic acid | 189 | 45 | 2.3e−39 |
| | | decarboxylase homolog. | | | |
| Methanococcus | Q57566 | Probable aromatic acid | 184 | 45 | 1.2e−38 |
| jannaschii | | decarboxylase (EC 4.1.1.—). | | | |
| Thermoplasma | Q9HJ72 | Probable aromatic acid | 180 | 45 | 1.5e−35 |
| acidophilum | | decarboxylase (EC 4.1.1.—). | | | |
| Methanobacterium | O26250 | Probable aromatic acid | 191 | 44 | 6.2e−37 |
| thermoautotrophicum | | decarboxylase (EC 4.1.1.—). | | | |
| Methanococcus | Q6LX02 | Flavoprotein:Phenylacrylic | 185 | 44 | 3.9e−35 |
| maripaludis | | acid decarboxylase, 3- | | | |
| | | octaprenyl-4- | | | |
| | | hydroxybenzoate carboxylyase | | | |
| | | (EC 4.1.1.—). | | | |
| Bacillus halodurans | Q9KCC2 | Probable aromatic acid | 206 | 43 | 8.4e−33 |
| | | decarboxylase (EC 4.1.1.—). | | | |
| Pseudomonas | Q3K681 | Phenylacrylic acid | 211 | 43 | 4.6e−32 |
| fluorescens | | decarboxylase precursor. | | | |
| Neisseria meningitidis | Q9JXP4 | Probable aromatic acid | 189 | 42 | 1.1e−39 |
| | | decarboxylase (EC 4.1.1.—). | | | |
| Neisseria meningitidis | Q9JW78 | Probable aromatic acid | 189 | 42 | 2.3e−39 |
| | | decarboxylase (EC 4.1.1.—). | | | |
| Pseudomonas | Q9HX08 | Probable aromatic acid | 209 | 42 | 4.6e−32 |
| aeruginosa | | decarboxylase (EC 4.1.1.—). | | | |
| Dechloromonas | Q47K05 | Phenylacrylic acid | 203 | 42 | 2.6e−31 |
| aromatica | | decarboxylase. | | | |
| Deinococcus radiodurans | Q9RR91 | Probable aromatic acid | 195 | 41 | 2.8e−34 |
| | | decarboxylase (EC 4.1.1.—). | | | |
| Sulfolobus solfataricus | Q9Y8K8 | Probable aromatic acid | 201 | 41 | 2.8e−32 |
| | | decarboxylase (EC 4.1.1.—). | | | |
| Nitrosococcus oceani | Q3J8P2 | Phenylacrylic acid | 205 | 41 | 1.1e−30 |
| | | decarboxylase. | | | |
| Vibrio cholerae | Q9KP38 | Probable aromatic acid | 211 | 41 | 1.0e−27 |
| | | decarboxylase (EC 4.1.1.—). | | | |
| Chlamydia pneumoniae | Q9Z8S4 | Probable aromatic acid | 192 | 40 | 1.4e−32 |
| | | decarboxylase (EC 4.1.1.—). | | | |
| Thiobacillus denitrificans | Q3SLD4 | Phenylacrylic acid | 205 | 40 | 1.8e−30 |
| | | decarboxylase precursor. | | | |
| Bacillus pseudofirmus | P94300 | Probable aromatic acid | 200 | 40 | 2.9e−30 |
| | | decarboxylase (EC 4.1.1.—). | | | |
| Desulfuromonas | Q40MZ3 | Phenylacrylic acid | 201 | 39 | 1.8e−30 |
| acetoxidans | | decarboxylase. | | | |
| Anabaena variabilis | Q3MFR2 | Phenylacrylic acid | 207 | 39 | 9.9e−30 |
| | | decarboxylase. | | | |
| Helicobacter hepaticus | Q7VGG1 | Phenylacrylic acid | 197 | 39 | 1.5e−26 |
| | | decarboxylase. | | | |
| Aquifex aeolicus | O66811 | Probable aromatic acid | 189 | 38 | 2.6e−29 |
| | | decarboxylase (EC 4.1.1.—). | | | |
| Synechococcus | Q8DK39 | 3-octaprenyl-4- | 210 | 38 | 3.4e−29 |
| elongatus | | hydroxybenzoate carboxylyase. | | | |
| Synechocystis sp. | P72743 | Probable aromatic acid | 206 | 38 | 4.4e−27 |
| | | decarboxylase (EC 4.1.1.—). | | | |
| Chlamydia muridarum | Q9PKH2 | Probable aromatic acid | 192 | 37 | 2.3e−30 |
| | | decarboxylase (EC 4.1.1.—). | | | |
| Chlamydia trachomatis | O84222 | Probable aromatic acid | 192 | 37 | 1.6e−29 |
| | | decarboxylase (EC 4.1.1.—). | | | |
| Aeropyrum pernix | Q9YBF0 | Probable aromatic acid | 197 | 37 | 3.4e−29 |
| | | decarboxylase (EC 4.1.1.—). | | | |
| Helicobacter pylori | Q9ZJE3 | Probable aromatic acid | 187 | 37 | 8.3e−26 |
| | | decarboxylase (EC 4.1.1.—). | | | |
| Helicobacter pylori | O26011 | Probable aromatic acid | 187 | 37 | 2.2e−25 |
| | | decarboxylase (EC 4.1.1.—). | | | |
| Campylobacter lari | Q4HKJ0 | Phenylacrylic acid | 193 | 37 | 7.4e−25 |
| | | decarboxylase, 3-octaprenyl- | | | |
| | | 4-hydroxybenzoate carboxylyase | | | |
| | | (EC 4.1.1.—). | | | |
| Leptospira interrogans | Q8F3R5 | Probable aromatic acid | 204 | 36 | 9.5e−25 |
| | | decarboxylase (EC 4.1.1.—). | | | |
| Chlamydophila caviae | Q823A9 | Phenylacrylic acid | 192 | 35 | 1.7e−32 |
| | | decarboxylase. | | | |
| Campylobacter | Q4HQF0 | Phenylacrylic acid | 184 | 35 | 9.2e−27 |
| upsaliensis | | decarboxylase, 3-octaprenyl- | | | |
| | | 4-hydroxybenzoate carboxylyase | | | |
| | | (EC 4.1.1.—). | | | |
| Streptomyces coelicolor | Q9KYP1 | Probable aromatic acid | 216 | 35 | 2.4e−24 |
| | | decarboxylase (EC 4.1.1.—). | | | |
| Sulfolobus acidocaldarius | Q4JAF6 | Phenylacrylic acid | 220 | 34 | 3.1e−26 |
| | | decarboxylase (EC 4.1.1.—). | | | |
| Campylobacter jejuni | Q9PPF1 | Probable aromatic acid | 187 | 34 | 1.4e−23 |
| | | decarboxylase (EC 4.1.1.—). | | | |
| Campylobacter coli | Q4HI63 | Phenylacrylic acid | 187 | 32 | 8.8e−22 |
| | | decarboxylase, 3-octaprenyl- | | | |
| | | 4-hydroxybenzoate carboxylyase | | | |
| | | (EC 4.1.1.—). | | | |

Accesion number = EMBL Protein accession number.
Length = Protein length in amino acids
Identity % = Percentage amino acid identity against S. cerevisiae Pad1p amino acid sequence
E value = Expectation value. The number of different alignments with scores equivalent to or better than S score (a measure of the similarity of the query to the sequence shown) that are expected to occur in a database search by chance. The lower the E value, the more significant the score.

### Example 3

This example concerns the selection of PAD1-gene containing yeast in broth culture by 4-Fluorocinnamic acid.

Yeast strains S. cerevisiae BY4741 (PAD1) and S. cerevisiae YO5833 (Δ pad1) Euroscarf—http://web.uni-frankfurt.de/fb15/mikro/euroscarf/ were separately inoculated at 10³ cells/ml into 100 ml conical flasks containing 50 mls buffered YEPD pH 4.0 (glycerol 20 g/l, Bacteriological peptone 20 g/l, yeast extract 10 g/l, succinic acid 3 g/l, adjusted to pH 4.0 with potassium hydroxide) each containing higher concentrations of 4-Fluorocinnamic acid, 0-0.225 mM. Flasks were cultured at 25° C. for 14 days, shaken at 120 rpm on an orbital shaker (25 mm orbit). After 14 days, yeast growth was measured by spectrophotometer at 600 nm (Optical Density, OD 600 nm). At pH 4.0, 4-Fluorocinnamic acid, showed good selection for PAD1-containing yeasts over the concentration range 0.15-0.225 mM (FIG. 6).

### Example 4

This example concerns the selection of PAD1-gene containing yeast in broth culture by 4-Methylcinnamic acid.

Yeast strains S. cerevisiae BY4741 (PAD1) and S. cerevisiae YO5833—Euroscarf (Δ pad1) were separately inoculated at 10³ cells/ml into 100 ml conical flasks containing 50 mls buffered YEPD pH 4.0 (glycerol 20 g/l, Bacteriological peptone 20 g/l, yeast extract 10 g/l, succinic acid 3 g/l, adjusted to pH 4.0 with potassium hydroxide) each containing higher concentrations of 4-Methylcinnamic acid, 0-0.11 mM. Flasks were cultured for 14 days at 25° C., shaken at 160 rpm on an orbital shaker (25 mm orbit). After 14 days, yeast growth was measured by spectrophotometer at 600 nm (Optical Density, OD 600 nm). At pH 4.0, 4-Methylcinnamic acid, showed good selection for PAD1-containing yeasts over the concentration range 0.075-0.11 mM (FIG. 7).

### Example 5

This example concerns the selection by 4-Methylcinnamic acid in agar for PAD1-containing yeast cells.

Buffered YEPD containing 4-Methylcinnamic acid, was prepared as follows: glycerol 20 g/l, Bacteriological peptone 20 g/l, yeast extract 50 g/l, agar 15 g/l, succinic acid 3 g/l, 4-Methylcinnamic acid 15.55 mg/l (0.096 mM). Agar was sterilised by autoclaving at pH 6.0, cooled to 50° C., adjusted to pH 4.0, then poured into Petri dishes and allowed to set. Agar plates were spread with dilute cultures of S. cerevisiae BY4741 (PAD1) (upper half of plate—FIG. 8), and dilute cultures of S. cerevisiae YO5833—Euroscarf (Δ pad1) on the lower half of the plate. After 10 days incubation at 25° C., PAD1-containing yeast cells had formed large colonies on the upper half of the plate, while Δ pad1 yeast growth was barely perceptible (FIG. 8).

This example demonstrates the selection by 4-Methylcinnamic acid in agar for PAD1-containing yeast.

### Example 6

This example concerns the selection of transformed cells of S. cerevisiae using a PAD1-containing vector, plated onto 4-Methylcinnamic acid agar.

PAD1 was inserted into a 2 μm-based, multi-copy, episomal vector (pVTU 260-Euroscarf) between the constitutive ADH1 promoter and terminator sequences. pVTU 260 also contains an AmpR and a URA3 sequence conferring ampicillin resistance in bacteria and uracil prototrophy in yeast. Yeast strain S. cerevisiae YO5833 (Δ pad1, ura3) was transformed with pVTU 260-PAD1 by electroporation.

The electroporated culture was plated out onto 4-Methylcinnamic acid agar (glycerol 20 g/l, Bacteriological peptone 20 g/l, yeast extract 30 g/l, agar 15 g/l, succinic acid 3 g/l, 4-Methylcinnamic acid 15.55 mg/l (0.096 mM) and incubated for 10 days at 25° C. Large colonies (putative PAD1-containing transformants) were then re-streaked onto agar lacking uracil, for confirmation of selection. This medium contained Yeast Nitrogen Base 6.7 g/l, glucose 20 g/l, agar 15 g/l, leucine 60 mg/l and histidine 10 mg/l. Agar plates were incubated for 2 days at 25° C. FIG. 9 shows positive growth of a transformant colony restreaked onto uracil-free agar (left). A control colony, electroporated without plasmid, showed no growth on uracil free agar (right). Therefore the large colony selected on 4-Methylcinnamic acid agar was also a uracil prototrophy, brought about by transformation with the pVTU 260 vector containing both PAD1 and URA3.

This example demonstrates directly the selection of transformants by PAD1 on 4-Methylcinnamic acid agar.

1. Use of a nucleic acid sequence encoding a decarboxylation enzyme as a positive selectable marker in a recombinant prokaryote or yeast.

2. A method of selecting a recombinantly transformed prokaryote or yeast comprising the steps of:
a) transforming a prokaryote or yeast with a selectable marker comprising a recombinant nucleic acid sequence encoding a decarboxylation enzyme such that the decarboxylation enzyme is expressed by the prokaryote or yeast; and
b) exposing the prokaryote or yeast to a weak acid or a salt thereof at a concentration capable of inactivating the micro organism without the selectable marker.

3. A method according to claim 2 wherein the weak acid has a pKa of between pH 2.5 and pH 5.5, preferably between pH 4 and pH 5.

4. A method according to claim 3 wherein the weak acid is an acid selected from Table 1 or 2.

5. A method according to claim 2 wherein step b) is carried out at less than pH 6, preferably less than pH 5, more preferably less than pH 4.

6. (canceled)

7. A method according to claim 2 wherein the weak acid is a monocarboxylic acid.

8. A method according to claim 2 wherein the weak acid has a structure in accordance with formula (I) 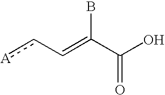
wherein [symbol] is:
(a) [symbol] alkyl optionally substituted; or
(b) an unsaturated aromatic group, optionally substituted with one or more hydrophobic groups; and
wherein B is hydrogen or a hydrophobic group.

9. A method according to claim 8, wherein the unsaturated aromatic group is selected from the group consisting of a phenyl group, a furanyl group, a thienyl group, pyrrole, cyclohexene, cyclopentene, cycloheptene, and pyridine.

10. A method according to claim 8, wherein the or each hydrophobic group is independently selected from: the group consisting of —F; —Cl; —Br; —I; C₁₋₃ alkyl; C₁₋₃ alkoxy; mono- di- or tri-fluoro C₁₋₃ alkyl; mono- di- or tri-chloro C₁₋₃ alkyl; mono- di- or tri-bromo C₁₋₃ alkyl; and mono- di- or tri-iodo C₁₋₃ alkyl.

11. (canceled)

12. Use according to claim 1 wherein the yeast is Saccharomyces cerevisiae.

13. Use according to claim 1 wherein the nucleic acid sequence is part of a vector which further comprises a promoter for controlling expression of the decarboxylation enzyme.

14. A vector comprising a nucleic acid sequence encoding a decarboxylation enzyme and a constitutive promoter for controlling expression of the decarboxylation enzyme.

15. (canceled)

16. (canceled)

17. (canceled)

18. (canceled)

19. (canceled)

20. (canceled)

21. (canceled)

22. (canceled)

23. A use according to claim 1, wherein the decarboxylation enzyme has at least 30% identity to SEQ. ID NO. 2.

24. A use according to claim 23 wherein the decarboxylation enzyme has at least 32%, 40%, 50%, 60%, 70%, 80%, 90% or 100% identity to SEQ. ID NO. 2.

25. (canceled)

26. (canceled)

27. (canceled)

28. (canceled)

29. Use of a decarboxylation enzyme as a selectable marker in a recombinant prokaryote or yeast.

30. (canceled)

31. (canceled)

32. (canceled)

33. A method according to claim 2, wherein the yeast is Saccharomyces cerevisiae.

34. A method according to claim 2, wherein the nucleic acid sequence is part of a vector which further comprises a promoter for controlling expression of the decarboxylation enzyme.

35. A method according to claim 2, wherein the decarboxylation enzyme has at least 30% identity to SEQ. ID NO. 2.

36. A method according to claim 35 wherein the decarboxylation enzyme has at least 32%, 40%, 50%, 60%, 70%, 80%, 90% or 100% identity to SEQ. ID NO. 2.

37. A vector according to claim 14, wherein the decarboxylation enzyme has at least 30% identity to SEQ. ID NO. 2.

38. A vector according to claim 37 wherein the decarboxylation enzyme has at least 32%, 40%, 50%, 60%, 70%, 80%, 90% or 100% identity to SEQ. ID NO. 2.


SEQUENCE LISTING (2 sequences)

SEQ ID NO: 1 - DNA, 729 bp, Saccharomyces cerevisiae
  CDS (1)..(729)
atgctcctatttccaagaagaactaatatagcctttttcaaaacaacaggcatttttgctaattttcctttgctaggtagaaccattacaacttcaccatctttccttacacataaactgtcaaaggaagtaaccagggcatcaacttcgcctccaagaccaaagagaattgttgtcgcaattactggtgcgactggtgttgcactgggaatcagacttctacaagtgctaaaagagttgagcgtagaaacccatttggtgatttcaaaatggggtgcagcaacaatgaaatatgaaacagattgggaaccgcatgacgtggcggccttggcaaccaagacatactctgttcgtgatgtttctgcatgcatttcgtccggatctttccagcatgatggtatgattgttgtgccctgttccatgaaatcactagctgctattagaatcggttttacagaggatttaattacaagagctgccgatgtttcgattaaagagaatcgtaagttactactggttactcgggaaacccctttatcttccatccatcttgaaaacatgttgtctttatgcagggcaggtgttataatttttcctccggtacctgcgttttatacaagacccaagagccttcatgacctattagaacaaagtgttggcaggatcctagactgctttggcatccacgctgacacttttcctcgttgggaaggaataaaaagcaagtaa

SEQ ID NO: 2 - protein, 242 aa, Saccharomyces cerevisiae
MLLFPRRTNIAFFKTTGIFANFPLLGRTITTSPSFLTHKLSKEVTRASTSPPRPKRIVVAITGATGVALGIRLLQVLKELSVETHLVISKWGAATMKYETDWEPHDVAALATKTYSVRDVSACISSGSFQHDGMIVVPCSMKSLAAIRIGFTEDLITRAADVSIKENRKLLLVTRETPLSSIHLENMLSLCRAGVIIFPPVPAFYTRPKSLHDLLEQSVGRILDCFGIHADTFPRWEGIKSK